(19) United States
(12) Patent Application Publication
(10) Pub. No.: US 2005/0015070 A1
(43) Pub. Date: Jan. 20, 2005

(54) Dialysis bag, a dialysis set comprising the bag, and a three-way connector for access to a dialysis bag

(75) Inventors: Annalisa Delnevo, Sant' Agata Bolognese (IT); Carlo Giannella, Mirandola (IT); Gianni Paltrinieri, Medolla (IT)

Correspondence Address: Finnegan, Henderson, Farabow,;Garrett & Dunner, L.L.P., 1300 I Street, N.W., Washington, DC, 20005-3315, US

(73) Assignees: GAMBRO DASCO S.p.A.; GAMBRO LUNDIA AB.

(21) Appl. No.: 10/885,639

(22) Filed: Jul. 8, 2004

Related U.S. Application Data
(60) Provisional application No. 60/511,316, filed on Oct. 16, 2003.

(30) Foreign Application Priority Data
Jul. 14, 2003 (IT) MO2003 A 000204

(51) Int. Cl. A61B 19/00

(52) U.S. Cl. 604/408

(57) ABSTRACT

A drainage bag (3), part of a dialysis set, comprises a wrapper (4), fluid-sealed, and a transparent pocket (10) superposed on a part of the wrapper (4); an edge of the pocket (10) has a first part (11a) which is fixed to the wrapper (4), and a second part (11b) which is free and delimits an opening of the pocket (10). The pocket (10) at least partially contains a drainage tube of the used dialysis liquid which is fluidly connected with an inside of the bag (3). The bag (3) keeps the tubing forming the dialysis set in a stable conformation both during a production process thereof and at moment of use, reducing a risk of endoluminal contamination of the dialysis set. The bag (3) is usable in particular for collection of a used and used peritoneal dialysis liquid.

### BACKGROUND OF THE INVENTION

The invention relates to a dialysis bag, a dialysis set comprising the bag and a three-way connector for access to a dialysis bag.

Specifically, though not exclusively, the invention is usefully applied in realising a drainage bag for peritoneal dialysis.

As is known, in peritoneal dialysis a defined quantity of sterile dialysis liquid is infused into the abdominal cavity—through a catheter made of a synthetic material previously applied to the patient—where it stays for some time, before being removed and discharged into a drainage bag.

Specifically, though not exclusively, the invention can be usefully applied in a peritoneal dialysis set comprising at least two bags, i.e. a supply bag full of fresh dialysis liquid and an empty drainage bag destined to receive the used dialysis liquid; the set further comprises a group of at least three portions of tube joined by a Y connection, in which a first portion of tube is for supplying the fresh dialysis liquid, a second portion of tube is for draining the spent dialysis liquid, and a third portion of tube is for connecting the catheter to the patient.

Usually the first and second portions of tube are connected to the supply bag and, respectively, to the drainage bag, during production of the set, while the third portion of tube is connected to the catheter at moment of use of the set.

Patent documents U.S. Pat. No. 5,053,003, U.S. Pat. No. 4,306,976, FR 2539034, EP 0116259 teach some examples of peritoneal dialysis sets of the above-described type.

This type of peritoneal dialysis set is supplied to the user closed in a sterile packaging, comprising a wrapping containing the various above-mentioned elements. Sometimes a further internal wrapping is supplied (called an inner-wrapping) which contains the empty drainage bag and the various portions of tube with relative connectors, which is for separating these articles from the full bag of sterile dialysis liquid, for a reason that will become clearer herein below. At moment of use, the user first opens the outer wrapping and performs the tasks necessary for setting up the dialysis set in the operative configuration; in particular, the user will open the inner wrapping, connect up the third portion of tube to the catheter and position the bags so that the liquid can move by force of gravity.

The various manoeuvres for readying the dialysis set can be laborious and not easy to perform, especially when the user has motor, visual or learning difficulties. In particular the manipulation of the various portions of tube can be difficult; once the packaging has been opened (both the outer and the inner wrappings) they are free and sometimes get tangled and are not easy to untangle and handle. Especially, during the package opening stage, there is the possibility that some parts of the dialysis set, for example the free ends of the various portions of tube, provided with connectors for connection to the catheter and the dialysis liquid supply bag, might come inadvertently into contact with non-sterile objects, considerably increasing the risk of contamination of the set (especially endoluminal contamination) with a consequent risk of onset of peritonitis.

A further drawback of peritoneal dialysis sets of known type concerns maintaining the various portions of tube in a stable configuration and as compact as possible, during the production process, especially during the stages of packing and sterilisation. The internal polypropylene wrapping is included for exactly this purpose; however the presence of an inner wrapping, which at moment of use must be opened by the user, causes a further complication in the setting up manoeuvres for the dialysis set.

The polypropylene inner wrapping also has a second purpose, i.e. avoiding direct contact with the portions of tube and the empty drainage bag with the full supply bag: this is to reduce the risk of there being surfaces in contact (especially PVC surfaces) which, by effect of heating during the sterilisation stage (generally steam) of the packaging, might stick together and make the dialysis set more difficult to use, especially during the setting-up stage, or even unusable.

### SUMMARY OF THE INVENTION

An aim of the present invention is to provide a drainage bag which obviates the above-described drawbacks in the prior art.

A further aim of the invention is to provide a dialysis set, in particular for peritoneal dialysis, which requires simple and practical manoeuvres on the part of the user for setting up in the use configuration.

An advantage of the invention is that it reduces the risk of endoluminal contamination of the dialysis set.

A further advantage of the invention is that it reduces the risk that some parts of the dialysis set might stick together by effect of heating up during the set sterilisation stage.

A further advantage is that the invention provides a dialysis set, in particular for peritoneal dialysis, which is simple and economical.

A still further advantage is that the entirety of tubes forming the dialysis set are kept stable and compact, both during the production process thereof and at moment of use.

Finally, a further advantage is that the invention provides a peritoneal dialysis set, a production process of which is simplified with respect to the prior art.

These aims and advantages and more besides are all achieved by the invention, as it is characterised in one or more of the accompanying claims.

In an embodiment of the invention, a dialysis bag, in particular for peritoneal dialysis, is provided with a lateral pocket for containing at least partially a tube or group of tubes for fluid connection with the bag during the dialysis treatment.

In a further embodiment of the invention, the tube is made of a transparent plastic material which is not PVC or which does not contain PVC or other chlorine.

In a further embodiment of the invention, the lateral pocket has an opening facing towards a fluid access port of the bag, in order to reduce the distance travelled by a tube between the access port to the bag and the access opening to the pocket.

In a further embodiment of the invention, a dialysis set, in particular for peritoneal dialysis, comprises: a drainage bag, for collecting used dialysis liquid and provided with a lateral pocket; a group of tubes, at least partially contained in the lateral pocket, at least one of which tubes is connected to the drainage bag; and possibly a bag full of sterile dialysis liquid, connected or to be connected at moment of use to at least one of the tubes.

In an embodiment of the invention, a connector, provided for connecting a liquid transport tube with a liquid collection bag, comprises a hollow body, provided with at least three ports, of which a first port is predisposed for connection with the transport tube, a second port is predisposed for introduction of the liquid into the collection bag, and a third port is predisposed for extraction of samples of liquid from the bag, the second and third ports being coaxial or nearly coaxial, so that a slim and straight element, for example a sampling needle, can be introduced into the bag passing through the ports.

Further characteristics and advantages of the present invention will better emerge from the detailed description that follows, of at least one embodiment of the invention, illustrated purely by way of non-limiting example in the accompanying figures of the drawings.

### BRIEF DESCRIPTION OF THE DRAWINGS

The description will now be made with reference to the accompanying figures of the drawings, provided by way of non-limiting example, and in which:

FIG. 1 is a frontal view of a drainage bag for peritoneal dialysis, made according to the invention, connected to a group of tubes for fluid transport;

FIG. 2 is the bag of FIG. 1, in which, for a better understanding of the invention, a part of the containing pocket of the group of tubes has been removed;

FIG. 3 is a view from the right of FIG. 2;

FIG. 4 shows the drainage bag of the previous figures with the group of tubes arranged outside the bag;

FIG. 5 is a complete set for peritoneal dialysis, comprising both the group of the previous figures (the drainage bag and the tubes) and the bag full of sterile dialysis liquid, all packed in a sealed outer wrapping;

FIG. 6 is a detail of the connector connecting up an end of the drainage tube with the access port of the drainage bag;

FIG. 7 is a detail of the drainage bag comprising the access port;

FIG. 8 is a section according to line VIII-VIII of FIG. 7.

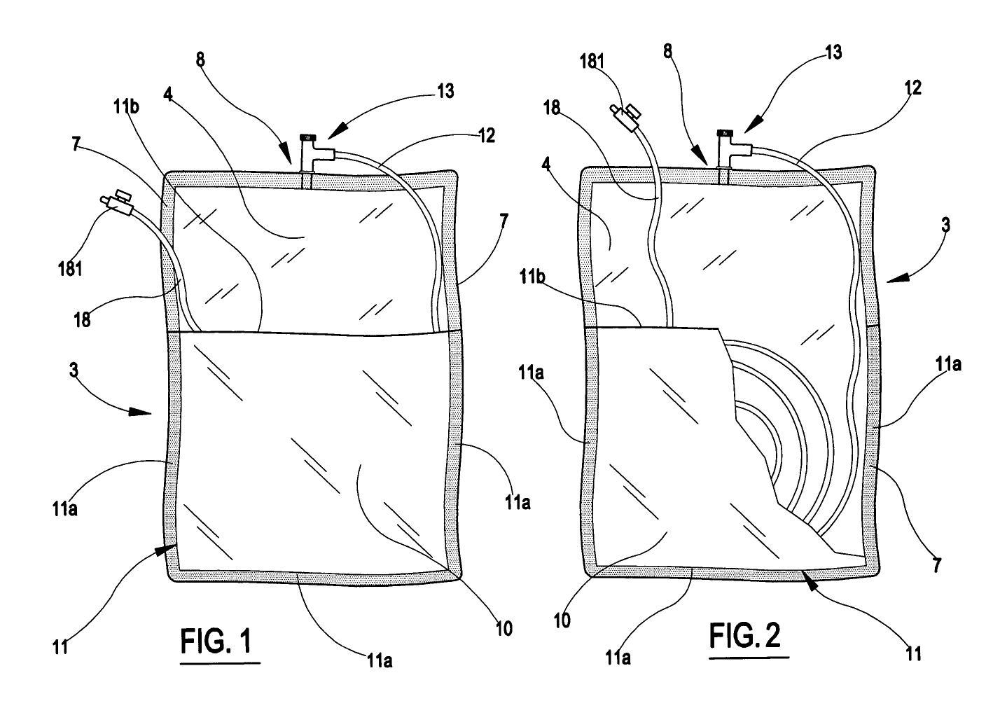

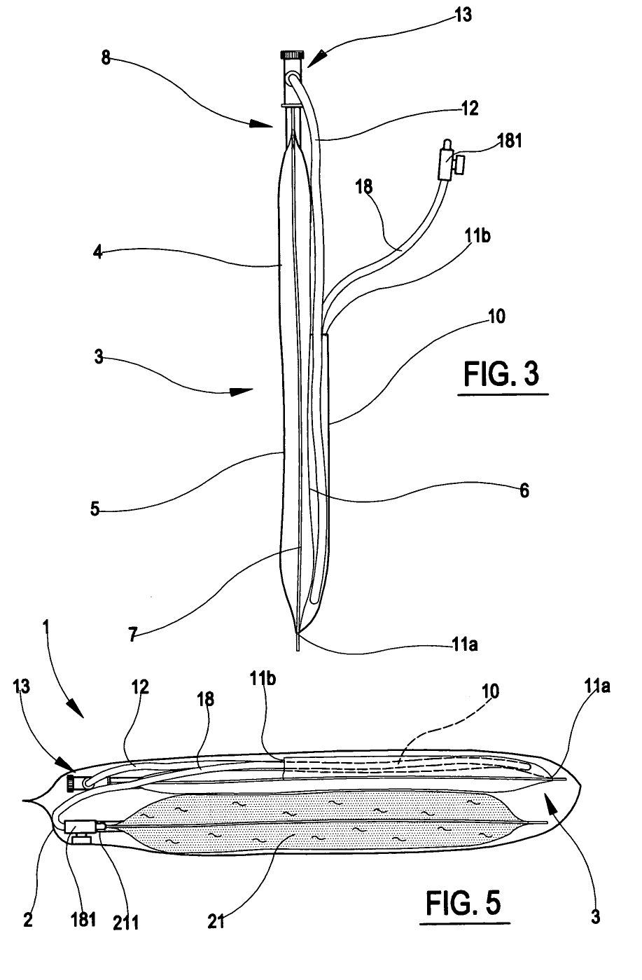

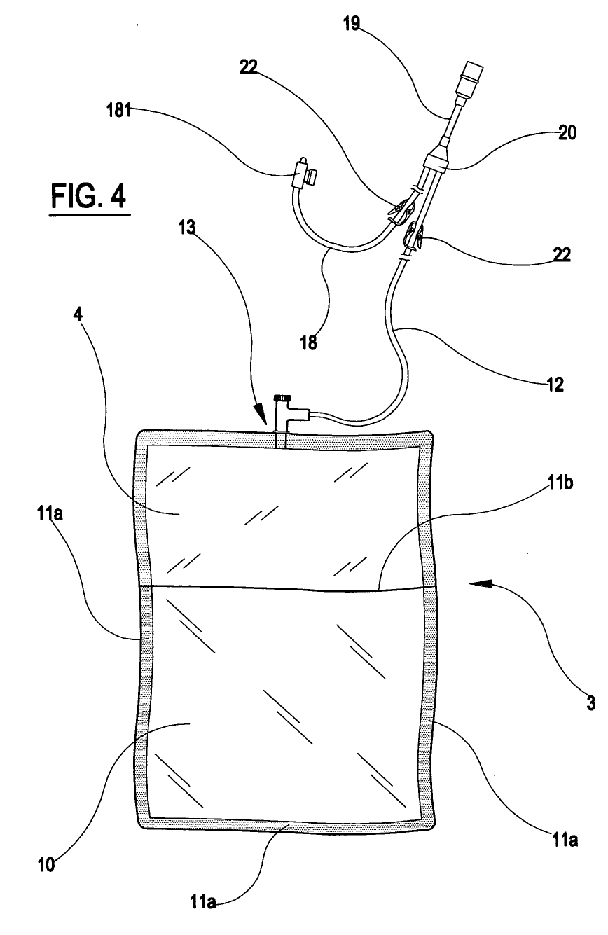

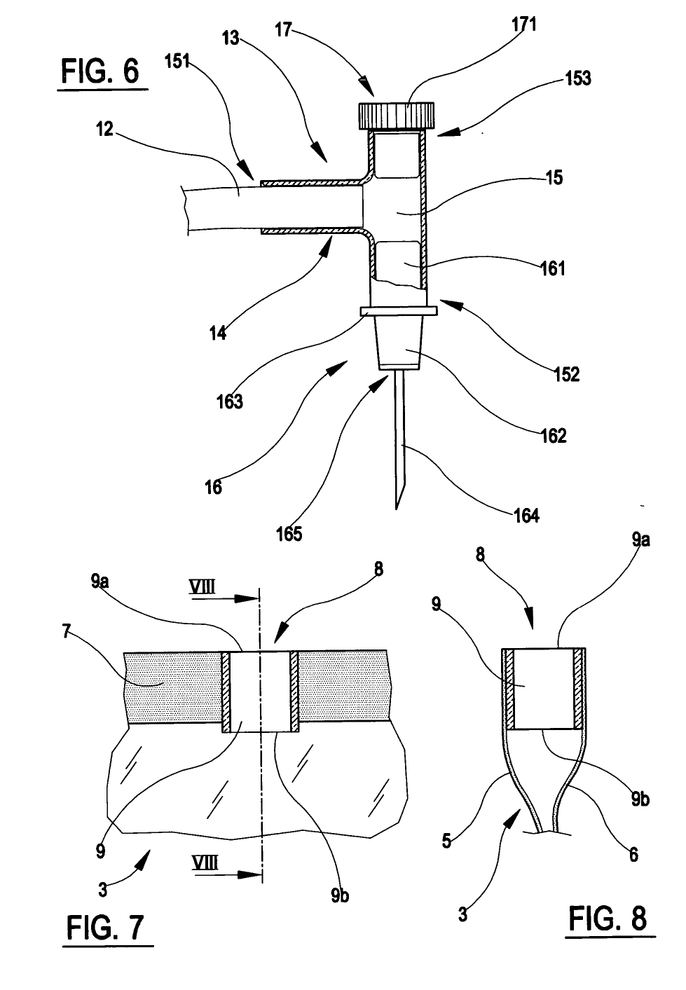

### DETAILED DESCRIPTION

1 denotes in its entirety (FIG. 5) a dialysis set, packed in a sealed envelope 2, ready for use. The description that follows relates in particular to a set which is suitable for peritoneal dialysis, although the invention is not limited solely to medical sets suitable for this type of dialysis.

The set 1 comprises a first bag 3 which is empty and which is a drainage bag for collecting used dialysis liquid coming from the abdominal cavity of the patient. The first bag 3 comprises an envelope 4, sealed and fluid-proof and having two flexible walls 5 and 6 superposed and joined one to another by welding along a perimeter 7 thereof. The envelope 4, flimsy when empty (as it appears at moment of use) defines the collection space which is destined to receive the used dialysis liquid. The perimeter 7 of the envelope 4 is, in the illustrated embodiment, rectangular, although it would be possible to have other perimeter shapes.

The envelope 4 is provided with an access port 8, for inlet of at least the used dialysis liquid, which access port 8 is a connector predisposed at the centre of a side of the perimeter 7 of the envelope 4 and destined for fluid connection with an end of a tube (the drainage tube of the used dialysis liquid).

The connector comprises a tubular element 9 (FIGS. 7 and 8) which is sealedly inserted between two opposite sheets of the envelope 4. The tubular element 9 is constituted by a short cylindrical tube, made of a flexible plastic material (for example a multi-layer material comprising at least one layer made of polypropylene and at least one layer made of PVC), which is held tight and fluid-proof between the two perimeter edges before the walls 5 and 6 of the envelope 4 are welded together. A first open end 9a of the tubular element 9 faces outwards of the envelope 4, while a second open end 9b, opposite to the first end 9a, faces inwards thereof.

The bag 3 is provided with a pocket 10 which is superposed on a part of a wall 6 of the wrapping. The pocket 10 has an edge 11, in the illustrated embodiment rectangular in shape, with a first part 11a which is fixed to the envelope 4 (for example by welding or gluing), and a second, free part 11b which is not fixed to the envelope 4. The first part 11a of the edge 11 of the pocket 10 comprises, in the illustrated embodiment, three sides of the rectangular perimeter of the edge 11, while the second part 11b comprises the fourth side. The free second part 11b defines an opening of the pocket 10 which is destined to receive, at least partially, one or more tubes which belong to the dialysis set, as will be better described herein below.

The first part 11a of the edge of the pocket 10, fixed to the envelope 4, develops at least partly parallel to the development of the perimeter 7 of the envelope 4. In particular, the pocket 10 is rectangular with sides which are parallel to the sides of the perimeter 7 of the envelope 4.

The opening of the pocket 10, delimited by the free second part 11b of the edge, faces towards the access port 8 of the envelope 4, so that a fluid transport tube, connected to the access port 8 of the envelope 4 and housed in the pocket 10, can follow a relatively short path in arriving from the access port 8 to the pocket 10.

The free second part 11b of the edge of the pocket develops between two opposite sides of the perimeter 7 of the envelope 4, in a transversal direction (in the illustrated embodiment, perpendicular) with respect to both the opposite sides. The access port 8 of the wrapping is predisposed on a zone of perimeter 7 of the wrapping comprised between the two above-mentioned opposite perimeter sides.

In particular the access port 8 is located on a perimeter side of the envelope 4, which perimeter side is parallel or nearly so, and distant from the second free part 11b of the pocket edge.

In the illustrated embodiment the pocket 10 occupies and covers about half the surface of one side of the envelope 4; in other embodiments (not illustrated) the dimensions of the pocket 10 with respect to the envelope 4 can be different, either bigger or smaller, than in the illustrated embodiment.

For example the pocket might occupy and cover more than half the surface of a side of the envelope 4, or cover it almost entirely, or even entirely.

The peritoneal dialysis set 2 further comprises a flexible first portion of tube 12 for transporting fluids for peritoneal dialysis, having a first end which is destined to be connected with the access port 8 of the drainage bag. The first portion of tube 12 is predisposed for drainage of a used dialysis liquid.

The first portion of tube 12 is fluidly and solidly connected to the access port 8 of the first bag, which is the collection bag for used liquid, by means of a three-way connector 13. The three-way connector 13 comprises a hollow body 14 provided with walls which delimit an internal cavity 15 therein. The hollow body 14 further exhibits three ports, all three in fluid communication with the internal cavity 15.

A first port 151 is predisposed for connection (for example by gluing using a solvent) with an end of the first portion of tube 12, a second port 152 is provided with a device 16 of known type for introduction of liquid into the access port 8 of the collection bag, and a third port 153 is provided with a fluid-proof sealed cap 17.

Each port 151, 152 and 153 comprises a tubular wall integrated with the above-mentioned hollow body 14. Each tubular wall comprises a cylindrical and smooth internal surface for coupling with the above-mentioned elements (the end of the first portion of tube 12, the device 16 for introduction of a liquid and the fluid-proof sealed cap 17).

The hollow body 14 is conformed in such a way that at least an imaginary straight line can be traced which crosses the above-mentioned internal cavity 15, entering by the third port 153 and exiting from the second port 152 without intersecting the walls of the hollow body 14. In the illustrated embodiment, the three ports 151, 152, 153 are arranged in a T-formation, and the second port 152 is coaxial and opposite to the third port 153. This formation of the ports 152 and 153 allows passage, through the two ports, of at least one straight line which does not touch the walls of the hollow body 14: this straight line is constituted, for example, by the common axis of the two ports 152 and 153. The function of this characteristic will emerge more clearly during the course of the present description.

The fluid-proof sealed cap 17, which is inserted (for example by gluing using a solvent) in the tubular wall of the third port 153, comprises a central part 171, of known type, which can be pierced by a needle, for introduction, through the access port 8 of the bag, of a needle for removing from the bag a sample of the liquid passing through the second and third port 152 and 153. Other access systems to the bag can be used, in particular for taking a liquid sample, such as, for example, a valve device, known as needle-less, of known type.

The special conformation and arrangement of the two ports 152 and 153, described above, allows easy access to the bag, for example by introducing, inside the envelope 4, at least a needle, or another slim and straight or practically straight object, passing through both the above-mentioned ports, the third port 153 and the second port 152.

In other words, a three-way connector 13 is couplable to the access port 8 of the bag, in which a first port is connectable to the liquid transport tube, a second port is connectable to the bag, faces inwardly of the bag itself and is arranged transversally to the first connector, and a third port is arranged in an opposite direction to the second connector. Thanks to this solution, the need to predispose a further access port (for passage of a liquid sampling needle) on the bag is avoided. In the solution proposed herein, the sampling needle can be introduced into the drainage bag directly through the access port with three-way connector, i.e. the same access port used for introduction of liquid coming from the drainage tube into the bag; this is thanks to the fact that the connector is provided with a third connector, sealed and safe, but which can be crossed by a needle if necessary, which third connector is opposite the second connector so that the second and third connectors together form a sort of single conduit which a needle can cross in passing from outside to inside the bag.

The device 16 for introduction of liquid in the bag comprises a first tubular portion 161 sealedly inserted (for example by gluing with a solvent) into the tubular wall of the second port 152, and a second tubular portion 162, sealedly inserted (for example by gluing with a solvent) into the tubular element 9 of the access port 8 of the drainage bag 3. A flange 163 is located between the two tubular portions 161 and 162, which flange has a larger diameter than the tubular portions 161 and 162, and is destined to be interpositioned between the second port 152 of the hollow body of the connector and the access port 8 of the bag.

The second tubular portion 162 bears a closure element 164, of known type, which at moment of use can be broken off to allow pouring of the liquid. The closure element 164, which is needle-shaped, is inserted, at least partially, into the access port 8 of the bag. The breaking of the closure element 164 determines the opening of the device 16, and thus allows the liquid to pour into the bag 3. The closure element 164, at a base thereof, exhibits a preferential break strip 165 (for example an annular weakening) to ease opening of the device 16.

The dialysis set 1 further comprises a second portion 18 of tube, which is flexible and predisposed for supply of a fresh dialysis liquid, and a third portion of tube 19, also flexible and predisposed for connection to a catheter of a patient.

The three portions of tube 12, 18 and 19, are united by a three-way connector 20, which can be Y-shaped and is of known type; the three-way connector has a first connector, connected to a second end, opposite the first end, of the first portion of tube 12, and a second and third connectors which are connected to the second portion of tube 18 and, respectively to the third portion of tube 19. The connecting-up of the three portions of tube 12, 18 and 19 to the connector is done in a known way.

The dialysis set 1 further comprises a second bag 21, full of fresh dialysis liquid, which is fluidly connectable to the second portion of tube 18 for fluid transport for peritoneal dialysis.

Normally the second portion of tube 18 is connected to the second bag 21, which is full, by insertion of an end 181 of the second portion of tube 18 in an access port 211, with easy-break closure, which the full bag 21 is provided with.

The envelope 4 and the pocket 10 are both made of materials, for example plastic materials, which do not cause sticking thereof to the portions of tube 12, 18 and 19 during a heating process of the production stage of the set, in particular during a steam-sterilisation stage. The envelope 4 and the pocket 10 are both made of plastic material not containing PVC. The pocket 10 can be made of plastic materials not containing PVC. The pocket 10 can be made of a transparent material, different to the material used to make the wrapping 4, which latter can also be transparent. In the illustrated embodiment, the envelope 4 is made of a multi-layer film, comprising at least one internal layer made of polypropylene and at least one external layer made of polyester or polyamide, and the pocket is made of polypropylene, or another polyolefin material, or of a polyolefin-based co-polymer, or polyamide, or a multi-layer film comprising two or more layers of the above-mentioned materials.

The pocket 10, integrated to the drainage bag 3, can contain, stably and compactly, the majority of the length of tubing for the dialysis set 1, both during the packaging stage of the dialysis set 1 in the external envelope 2, and during the sterilisation stage (for example by steam), and also during the following stages of transport, stocking, right up to the final stage of opening the package at moment of use, by the user.

During the final stage, in which the user breaks open the external envelope 2, the risk that the user might lose control of the tubing and that these might come into contact with non-sterile surfaces is reduced to a minimum, as the tubes stay still and protected inside the pocket 10. In this way the invention offers the advantage of reducing the risk of endoluminal contamination of the dialysis set 1.

After having opened the external envelope 2 and extracted the dialysis set 1 from the envelope 2, the user can see the set of tubes 12, 18 and 19 contained in the transparent pocket 10, and can extract them from the pocket unhurriedly and with no violent movements, simply by reaching in and pulling them out.

The tubes 12, 18 and 19 can be extracted from the pocket by removal of the pocket itself; in this case the pocket 10 can be coupled to the envelope 4 removably, for example by an easy-peel coupling to the wrapping.

The user can perform this removal operation very practically and easily, by gripping the free edge 11b of the pocket and pulling, thus breaking the pocket, or by detaching the edge 11a of the pocket from the envelope 4. In either case, the various tubes of the dialysis set 1 can exit the pocket with no special effort, and therefore under controlled circumstances.

The pocket 10 protects the various portions of tube 12, 18 and 19, and relative connectors and devices (for example, the clamps 22), even during some stages of the production process.

In particular, the pocket 10 protects the tubing of the dialysis set 1 during the steam-sterilisation stage, thus avoiding the risk of sticking between the parts of the set, in particular between the tubing (normally made of PVC) and the full bag 21 (which can also be made of PVC). For this purpose, the dialysis set 1 is packed, with the drainage bag 3 superposed on the full bag 21 (see FIG. 5)

By predisposing at least one pocket, integrated with the drainage bag, the dialysis set 1 can be realised using simple joining techniques for the plastic parts (welding, gluing, etc.); it thus offers the advantage of providing a peritoneal dialysis set 1 which is relatively simple and economical.

1. A bag for dialysis comprising:
a fluid-proof sealed envelope;
at least one access port predisposed on said envelope and provided with a connector designed to be fluidly connected to an end of a portion of tube for fluid transport;
a pocket at least partially superposed on at least a part of a wall of said envelope, said pocket being designed to receive, at least partially, said portion of tube.

2. The bag of claim 1, wherein at least an edge of said pocket has a first part which is engaged to said envelope, and a second part which is free and which delimits an access opening to said pocket.

3. The bag of claim 2, wherein said envelope comprises at least two flexible walls which are reciprocally superposed and joined along a perimeter, and wherein said first part of edge of pocket has a development which at least in part follows in a parallel direction the development of said perimeter.

4. The bag of claim 2, wherein said opening, delimited by said free second part of edge, is facing in a direction towards said access port.

5. The bag of claim 1, wherein said pocket is made of a transparent material.

6. The bag of claim 1, wherein said envelope and said pocket are made of materials which do not contain PVC.

7. The bag of claim 1, wherein said pocket is made of a material selected in the group consisting of: a polyolefin material, a polyolefin-based co-polymers, a polyamide, a multi-layer film consisting of at least two layers of the above-named materials.

8. The bag of claim 2, wherein said free second part of the edge of the pocket develops between two opposite perimeter sides of the envelope, in a transversal direction to said two opposite perimeter sides, said access port being predisposed in a perimeter zone of said envelope comprised between said two opposite perimeter sides.

9. The bag of claim 2, wherein said access port is located on a perimeter side of said envelope, said perimeter side being parallel or nearly parallel to said free second part of the edge of the pocket.

10. The bag of claim 1, wherein said envelope has a perimeter which is of rectangular shape, and wherein said pocket is also of rectangular shape, with sides parallel to sides of the perimeter of said envelope.

11. A bag for dialysis comprising:
a fluid-proof sealed envelope;
at least one access port predisposed on said envelope and designed to be fluidly connected to a tube for fluid transport;
a pocket associated to said envelope and designed to receive, at least partially, said tube.

12. A series of elements for dialysis, wherein:
a first element of the series comprises at least a first bag, made as the bag of claim 1 or claim 11;
a second element of the series comprises at least a first portion of tube for dialysis fluid transport, having at least a first end which is designed for connection with said access port of said first bag.

13. The series of elements of claim 12, wherein said first portion of tube is predisposed for draining a spent peritoneal dialysis fluid, and wherein said second element of the series further comprises:
at least a second portion of tube predisposed for supply of a fresh dialysis fluid;
at least a third portion of tube predisposed for connection to a catheter of a patient; and
at least one three-way connector for connecting up said first, second and third portions of tube.

14. The series of elements of claim 11, wherein a third element in the series of elements comprises a second bag, full of fresh dialysis fluid, which is fluidly connectable to at least a second portion of tube for fluid transport for dialysis.

15. A connector for connection of a fluid transport tube, for example a spent dialysis liquid, to a liquid collection bag, comprising a hollow body, provided with walls which delimit an internal cavity, and further provided with at least three ports all three of which are in fluid communication with said internal cavity, a first port of which being predisposed for connection with an end of said liquid transport tube, a second port being provided with a device for introduction of the liquid into an access port of said collection bag, a third port being provided with a fluid-sealed cap, said hollow body being conformed in such a way as to allow a tracing of at least one imaginary straight line crossing said internal cavity, entering by said third port and exiting from said second port, without intersecting the walls of said hollow body.

16. The connector of claim 15, wherein said second port is coaxial and opposite to said third port.

17. The connector of claim 15, wherein each of said three ports comprises a tubular wall which is an integral part of said hollow body.

18. The connector of claim 15, wherein said fluid-sealed cap comprises at least a part which can be perforated by a needle for introduction, into said access port of said bag, of a needle for removing a sample of liquid from the collection bag, passing through said second and third ports.

19. The connector of claim 15, wherein said fluid-sealed cap comprises a valve device of a needleless type for access to the bag, in particular for removing a sample of liquid.

20. The connector of claim 15, wherein said device for introducing liquid into the bag comprises a closure element which is breakable and which is designed to be at least partially inserted in said access port of the bag.

21. The connector of claim 19, wherein said device for introduction of liquid into the bag comprises at least a first tubular portion, sealedly inserted in a tubular wall of said second port, and at least a second tubular portion which bears said breakable closure element and which can be sealedly inserted in said access port of the bag.

22. The connector of claim 15, wherein said three ports are arranged in a T-formation.

23. A set for dialysis comprising:
at least one bag for containing a liquid, which bag comprises an envelope having at least one access port;
at least a first portion of tube for dialysis fluid transport, having at least a first end which is connected with said access port by means of the connector of claim 15.

24. The set of claim 23, wherein said bag is realised according to claim 1 or claim 11.

25. The set of claim 23, wherein said first portion of tube is predisposed for drainage of a spent dialysis fluid, and wherein the set further comprises:
at least a second portion of tube which is predisposed for supplying a fresh dialysis fluid;
at least a third portion of tube predisposed for connection to a catheter of a patient; and
at least a three-way connector, a first way thereof being connected to at least a second end, opposite to the first end, of said first portion of tube, a second and a third ways thereof being connected respectively to said second portion of tube and to said third portion of tube.

26. The set of claim 23, comprising a second bag which is full of a fresh dialysis liquid, which is fluidly connectable to at least a second portion of tube for dialysis fluid transport.
